(19) United States
(12) Patent Application Publication
(10) Pub. No.: US 2020/0029907 A1
(43) Pub. Date: Jan. 30, 2020

(54) SEATING SENSOR, SEAT, AND WAVEFORM ANALYSIS APPARATUS

(71) Applicant: TAIYO YUDEN CO., LTD., Chuo-ku, Tokyo (JP)

(72) Inventors: Keiichi KOBAYASHI, Takasaki-shi (JP); Takashi ISHIGURO, Takasaki-shi (JP)

(21) Appl. No.: 16/498,342

(22) PCT Filed: Mar. 12, 2018
(86) PCT No.: PCT/JP2018/009385

§ 371 Date: Sep. 26, 2019

(30) Foreign Application Priority Data
Mar. 29, 2017 (JP) 2017-064942

(51) Int. Cl. A61B 5/00; G01H 11/08; A61B 5/021

(52) U.S. Cl. CPC A61B 5/6891; G01H 11/08; A47K 13/24; A61B 5/6893; A61B 5/72; A61B 5/02108

(57) ABSTRACT

In an exemplary embodiment, a seating sensor 280 includes a sensor module 200 provided with a piezoelectric element 30 and attached to a seat 302 via a vibration transmission plate 210. When a user is seated on the seat 302, pulse wave vibrations due to blood flow of the user are transmitted through the seat 302 to its reverse face side, and then transmitted to the vibration transmission plate 210 and further transmitted, via a vibration ring 40, to the piezoelectric element 30 in the sensor module 200, whereupon a seating of the user on the seat 302 and/or the user's biological information are/is detected. The vibration transmission plate 210 allows the pulse wave vibrations from the user to be transmitted to the sensor module 200 even when the user does not sit on the seat 302 directly above the seating sensor 280.

### TECHNICAL FIELD

The present invention relates to application of a vibration waveform sensor that measures the waveforms of various types of vibrations such as pulses, and more specifically to a seating sensor, a seat, and a waveform analysis apparatus, for detecting seating condition or biological information such as pulse waves using a piezoelectric element.

### BACKGROUND ART

Among sensor devices that have been proposed for the purpose of managing health based on continuous measurement of pulses, are vibration waveform sensors that utilize piezoelectric elements. For example, the patent document mentioned below discloses a prior art that applies such a sensor for medical diagnosis, where the sensor is used in a loop wound around one's fingertip to capture the vibrations through the arterial vessel walls at the fingertip, as pulse waves.

FIG. 1(A) shows a vibration waveform sensor being used as a pulse wave sensor, where FIG. 1(A) shows a cross-sectional view, FIG. 1(B) shows an exploded view, and FIG. 1(C) shows a view from the bottom face side, of a sensor module 10. In these figures, the sensor module 10 is structured in such a way that a piezoelectric element 30 is placed on a principal face of a substrate 20 and this piezoelectric element 30 is covered with a vibration ring 40. The aforementioned piezoelectric element 30 is rectangular and has long sides, as shown in FIG. 1(C).

The substrate 20 is used to immovably support the piezoelectric element 30 while allowing its electrodes to be led out and its signals to be amplified, and has, on its principal face, a pair of electrode lands 22, 23 provided near the center and a ground conductor 24 formed around them. The electrode lands 22, 23 are led out to the reverse face side of the substrate 20 via through holes 22A, 23A. To the electrode lands 22, 23, terminals (not illustrated) of the piezoelectric element 30 are joined by a conductive adhesive, etc. This way, the piezoelectric element 30 is connected to an amplifier (described later), etc., provided on the reverse face side of the substrate 20 via the electrode lands 22, 23 and through holes 22A, 23A.

Next, the vibration ring 40 is provided in a manner encircling the aforementioned piezoelectric element 30, and the vibration ring 40 is electrically joined to the ground conductor 24. Also, the ground conductor 24 is led out to the reverse face side of the substrate 20 via through holes 24A, 24B (illustrated only in FIG. 1(A)). The vibration ring 40 has conductivity and achieves a common ground potential between it and the human skin it contacts, while at the same time functioning as a vibration introducer that introduces the vibrations of the skin and further transmits them to the substrate 20. The vibrations of the skin are transmitted to the vibration ring 40, while at the same time transmitted to the substrate 20 from the vibration ring 40. The substrate 20 also functions as a vibrator so that the vibrations transmitted from the vibration ring 40 are transmitted to the piezoelectric element 30. A cavity 41 is formed by this vibration ring 40.

The sensor module 10 described above is worn on a human fingertip using a medical fixing tape 12 in such a way that the vibration ring 40 contacts the human skin BD, as shown in FIG. 2(A). Pulse wave signals output from the piezoelectric element 30 of the sensor module 10 are amplified by an instrumentation amplifier 26, and then input to a mainboard 50. On the mainboard 50, the signals are amplified further by a programmable amplifier 52, after which they are converted to digital signals by an A/D converter 53 and then transmitted from a transmission module 54. The transmitted pulse wave signals are received by a reception module 62 in a USB dongle 60, and input to a waveform analysis apparatus 100 through a USB interface 64.

In the waveform analysis apparatus 100, the input data is stored in a data memory 110 as waveform data 112. When a noise elimination program 122 is run in a CPU 102, and any disturbance that exceeds a pre-set threshold occurs in the waveform data 112, the waveform is peak-held and the noise is eliminated. When a waveform diagnosis program 124 is run in the CPU 102, the waveform data 112 is calculated/analyzed and the calculation result is stored in the data memory 110 as calculation data 114 and also displayed on a display 104. Also, an arrythmia detection program 126 is run in the CPU 102 to detect arrythmia. Furthermore, if the aforementioned calculation result exceeds a threshold or arrhythmia is detected, an alert program 128 outputs a corresponding alert in the form of light or sound. The aforementioned constitutions and operations are disclosed in Patent Literature 1.

### BACKGROUND ART LITERATURE

### Patent Literature

Patent Literature 1: International Patent Laid-open No. 2016/167202

### SUMMARY OF THE INVENTION

### Problems to Be Solved by the Invention

The aforementioned pulse wave sensor can sense pulse waves other than at the fingertip; for example, it can be utilized as a seating sensor by assembling it into a chair to measure pulse waves at the thigh, because this allows for detection of whether or not a person is seated on the chair.

It should be noted that, when the vibration waveform sensor is assembled into a chair, pulse waves cannot be measured in a favorable manner unless the leg is placed exactly and directly above the sensor, which is different from the aforementioned case on the finger. This is because the vibrations (pulse waves) to be sensed by the aforementioned vibration waveform sensor are very small and, unless the sensor is positioned directly above or directly below an artery, pulse waves cannot be sensed. The sensing sensitivity drops significantly according to the distance from directly above or directly below the artery. Also, with a chair, the position relationship between the thigh or hip and the sensor varies based on the person's physical size or the position where the person is seated. As a result, stable measurement of pulse waves has been difficult even when the aforementioned vibration waveform sensor is assembled directly into a chair.

The present invention focuses on this point, and its object is to provide a seating sensor, a seat, and a waveform analysis apparatus, each offering a wider seating range in which pulse waves can be sensed, in order to reduce the effects of physical size and seating position and thereby detect seating/not-seating and biological information in a favorable manner.

### Means for Solving the Problems

The seating sensor proposed by the present invention represents a seating sensor that detects biological vibrations from a user of a seat by utilizing a sensor module comprising: a piezoelectric element that measures the vibrations of a substrate to obtain vibration waveforms; and a vibration introducer provided on the substrate, which contacts a target object and transmits its vibrations to the substrate; wherein such seating sensor is characterized in that a vibration transmission plate is provided on the seat, and the sensor module is fixed to the seat by a fixing means in such a way that the vibration introducer contacts this vibration transmission plate.

One preferred embodiment is characterized in that the vibration introducer is conductive. It is further characterized in that the vibration introducer is a conductive ring and the space inside the ring is filled with a resin. Another embodiment is characterized in that the vibration transmission plate makes surface contact with the seat and transmits vibrations from the person seated on the seat to the vibration introducer. It is further characterized in that the modulus of elasticity of the vibration transmission plate is 70 to 2500 MPa. Yet another embodiment is characterized in that the vibration introducer is pressed against the vibration transmission plate by the fixing means.

The seat proposed by the present invention is characterized in that it is equipped with any one of the aforementioned seat sensors. Also, the seat is characterized in that it encompasses chairs, seats, toilet seats, and others that can be sat on by a person. The waveform analysis apparatus proposed by the present invention is characterized in that it detects, from the detection signals of the piezoelectric element in any one of the aforementioned seating sensors, at least either one of: whether or not the user of the seat is seated on the seat, and biological information of the user. The aforementioned and other objects, features, and benefits of the present invention are made clear by the detailed explanations provided below and the drawings attached hereto.

### Effects of the Invention

According to the present invention, the vibration transmission plate is provided in the seat, and it is also fixed in such a way that the vibration introducer of the sensor module contacts this vibration transmission plate; this permits widening of the seating range in which pulse waves can be sensed, so that the effects of physical size and seating position can be reduced to allow for detection of seating/not-seating and biological information in a favorable manner.

### BRIEF DESCRIPTION OF THE DRAWINGS

FIG. 1 Drawings showing the vibration waveform sensor under the prior art, where (A) is a cross-sectional view, (B) is an exploded view, and (C) is a plan view seen from the mounting face side of the substrate.

FIG. 2 Drawings showing the configurations of a waveform analysis system using the aforementioned vibration waveform sensor, where (A) shows the overall device configuration, while (B) and (C) are block diagrams showing the circuit configuration.

FIG. 3 Drawings showing the configurations of the sensor module and seating sensor in an example of the present invention, where (A) shows a cross-section of the sensor module, while (B) and (C) show cross-sections of the seating sensor.

FIG. 4 Drawings showing examples of installation of the aforementioned seating sensor, where (A) and (B) represent an example of installation in a toilet seat, while (C) represents an example of installation in an automobile seat.

FIG. 5 Drawings showing examples of seating positions and detected pulse wave signals in the aforementioned example of installation in a toilet seat.

FIG. 6 Drawings showing examples of seating positions and detected pulse wave signals when no vibration transmission plate is provided.

### MODE FOR CARRYING OUT THE INVENTION

The best mode for carrying out the present invention is explained in detail below based on an example.

### EXAMPLE 1

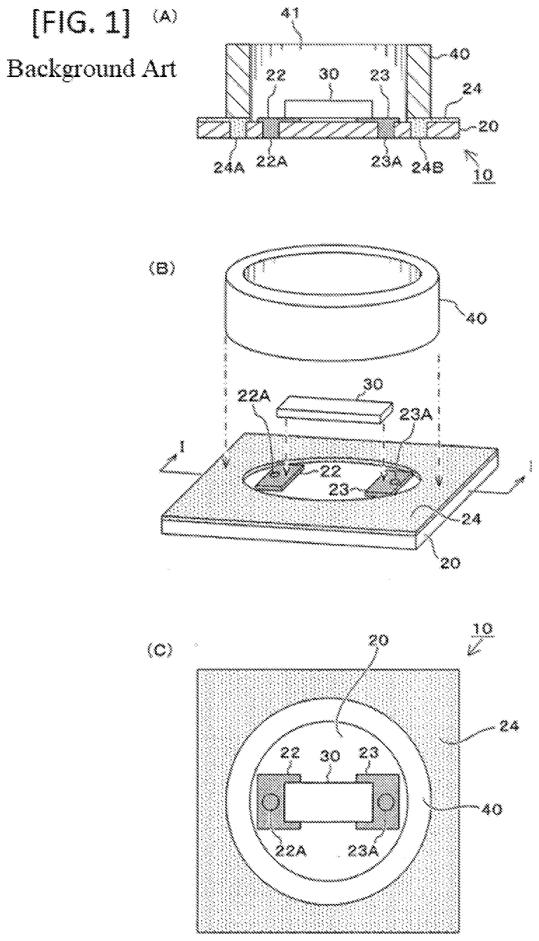

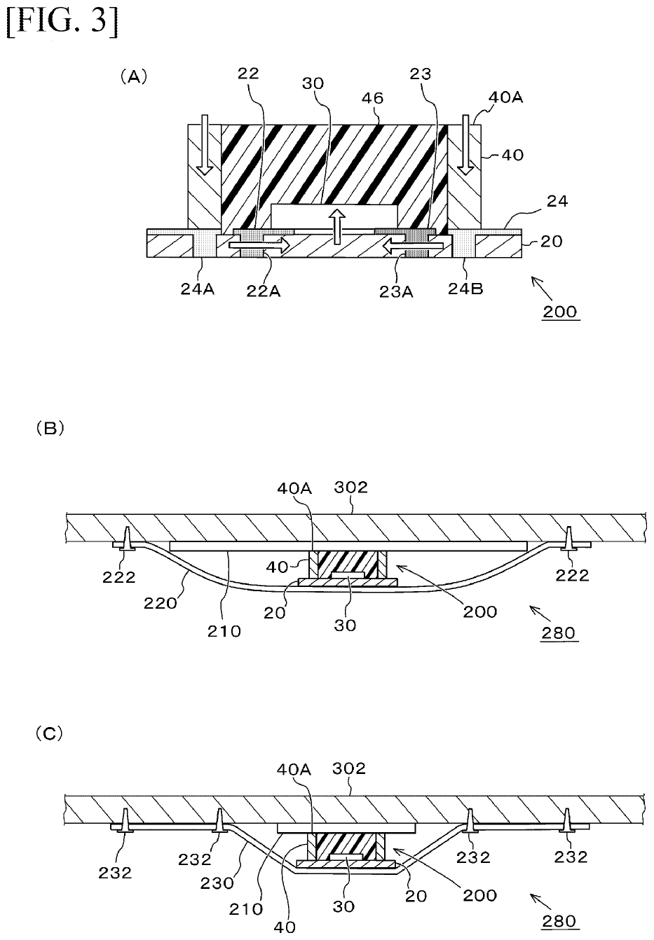

Example 1 of the present invention is explained below by referring to FIG. 3 to FIG. 6. As shown in FIG. 3(A), basically the sensor module 200 of the vibration waveform sensor in this example is structurally identical to the aforementioned counterpart shown in FIG. 1, where the structure is that a piezoelectric element 30 is placed on a principal face of a substrate 20 and this piezoelectric element 30 is covered with a vibration ring 40. On the principal face of the substrate 20, a pair of electrode lands 22, 23 are provided near the center, and a ground conductor 24 is formed around them. The electrode lands 22, 23 are led out to the reverse face side of the substrate 20 via through holes 22A, 23A. To the electrode lands 22, 23, terminals (not illustrated) of the piezoelectric element 30 are joined by a conductive adhesive, etc. This way, the piezoelectric element 30 is connected to an amplifier (described later), etc., provided on the reverse face side of the substrate 20 via the electrode lands 22, 23 and through holes 22A, 23A. Also, the vibration ring 40 is provided in a manner encircling the aforementioned piezoelectric element 30, and the vibration ring 40 is electrically joined to the ground conductor 24. Also, the ground conductor 24 is led out to the reverse-face side of the substrate 20 via the through holes 24A, 24B. The vibration ring 40 has conductivity and achieves a common ground potential between it and the human skin it contacts, while at the same time functioning as a vibration introducer that introduces the vibrations of the skin and further transmits them to the substrate 20. The vibrations of the skin are transmitted to the vibration ring 40, while at the same time transmitted to the substrate 20 from the vibration ring 40. The substrate 20 also functions as a vibrator so that the vibrations transmitted from the vibration ring 40 are transmitted to the piezoelectric element 30 (refer to the arrows in FIG. 3(A)).

In this example, a silicon resin 46 with high insulation property is filled in the cavity 41 that has been formed by the vibration ring 40. The resin may be filled to any degree as deemed appropriate, so long as a tip face 40A of the vibration ring 40 is exposed; however, it should not bulge out of the vibration ring 40, because the tip face 40A makes contact with the vibration transmission plate, as described later. Filling such silicone resin 46 protects the component and prevents shorting between the electrodes, while at the same time improving the moisture resistance and water resistance against humidity in air or sweat from the human body.

Among the above parts, for the substrate 20, a 12-mm square, 1-mm thick hard substrate made of glass epoxy, ceramic, etc., is used, for example. On the reverse-face side of this substrate 20, an amplifier or other electronic component is mounted. For the piezoelectric body constituting the piezoelectric element 30, PZT (lead zirconate titanate) is generally used; however, the material is not limited in any way and any material may be used so long as it has an appropriate sensitivity (piezoelectric constant, capacity), and as for the shape, any shape will do so long as it fits in a range of approx. 0.6×0.3 mm to 3.2×1.6 mm depending on the purpose of use. For the vibration ring 40, the material is not limited to a metal so long as it is hard and conductive, and it may be a hard plastic whose surface is plated with a metal, for example. Additionally, its shape need not be a ring, either, and it may be a square column whose two opposing sides alone are bonded. The vibration ring 40 causes the biological (pulse wave) vibrations from its tip face 40A to be transmitted reliably to the piezoelectric element 30, while at the same time allowing any electrical noise to be released to ground; accordingly, pulse wave signals of higher quality can be obtained.

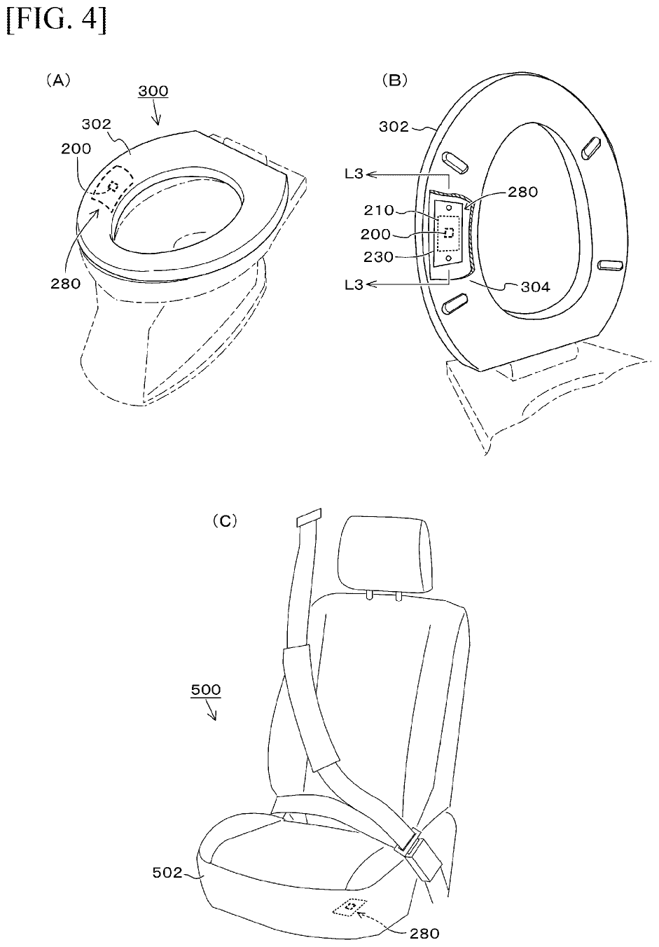

This sensor module 200 is installed on a toilet seat 302 of a toilet bowl 300, for example, as shown in FIG. 4 (A). FIG. 4 (B) is a view of the toilet seat 302 from the reverse-face side, while a view along line L3-L3 in this figure in the direction of the arrow is shown in FIG. 3 (B). As shown in these figures, the sensor module 200 is installed on the reverse-face side of the toilet seat 302 in such a way that the tip face 40A side of the vibration ring 40 comes in contact with the toilet seat 302 via a vibration transmission plate 210. The substrate 20 side of the sensor module 200 is widely covered by a fixing plate 220, and both ends of the fixing plate 220 are fixed to the toilet seat 302 by screws 222. These sensor module 200, vibration transmission plate 210, and fixing plate 220, as a whole, constitute a seating sensor 280.

Among the above parts, the toilet bowl 300 is widely known, and for the toilet seat 302, plastic types are generally used. In the illustrated example, the toilet seat 302 is structured in such a way that a space is formed between the top and bottom plastic plates, and the seating sensor 280 is installed on the reverse-face side of the top plastic plate inside this space. The vibration transmission plate 210 makes surface contact with the toilet seat 302 for collecting biological vibrations from the toilet seat 302 and transmitting them to the vibration ring 40, and is made by using a material that easily transmits vibrations (does not allow vibrations to attenuate easily) such as ABS, polyethylene resin, polycarbonate, or other resin material having a some degree of elasticity. To be more specific, preferably its modulus of elasticity is 70 to 2500 MPa and the Rockwell hardness is R20 to R180. If the modulus of elasticity is 70 MPa or less, the vibrations are absorbed, disabling vibration sensing in a favorable manner, which is not desirable; if it is 2500 MPa or more, on the other hand, the vibrations are absorbed at the interface with the resin part and the sensing sensitivity drops as a result, which is not desirable. Additionally, if the Rockwell hardness is R20 or less, the vibrations are also absorbed and cannot be sensed, which is not desirable; if it is R180 or more, on the other hand, similarly the vibrations are absorbed at the interface with the resin part and the sensing sensitivity drops as a result, which is not desirable.

For the fixing plate 220, a plastic, etc., may be used, but a rigid, thick hook-and-loop fastener tape, or adhesive tape may also be used, for example. When the sensor module 200 is pressed, the closeness of contact among the vibration ring 40, vibration transmission plate 210, and toilet seat 302 improves, allowing the biological vibrations to be transmitted in a favorable manner and improving the sensitivity. For the screws 222, truss screws, etc., having a wide base area are used, for example. An adhesive, etc., may also be used. FIG. 3(C) provides an example where screws 222 are added to increase the force by which the sensor module 200 is pressed against the toilet seat 302.

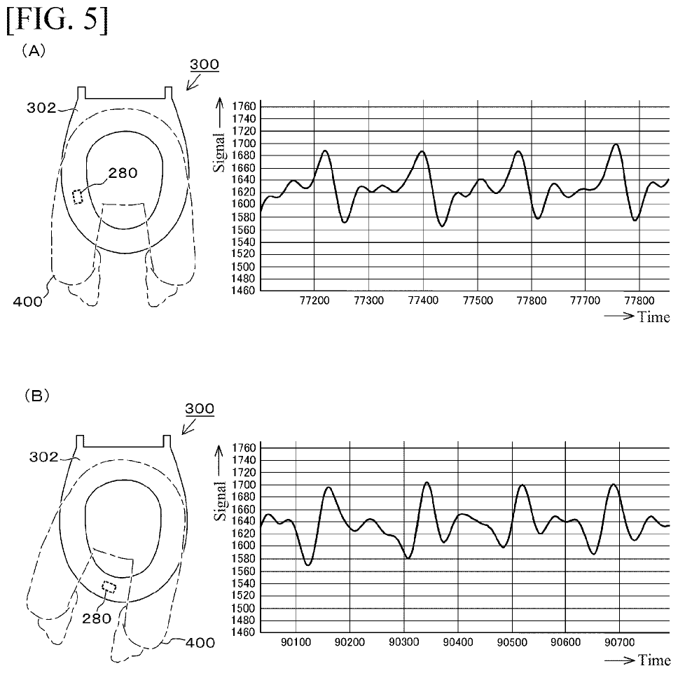

Next, the operation of the seating sensor 280 is explained by referencing FIG. 5(A) where, as shown, seating of the subject 400 on the toilet seat 302 results in a thigh of the subject 400 positioned directly above the seating sensor 280. As a result, the biological vibrations at the thigh, such as pulse wave vibrations due to blood flow, are transmitted through the toilet seat 302 to its reverse face side. These biological vibrations are transmitted to the vibration transmission plate 210 and further transmitted, via the vibration ring 40, to the piezoelectric element 30 in the sensor module 200.

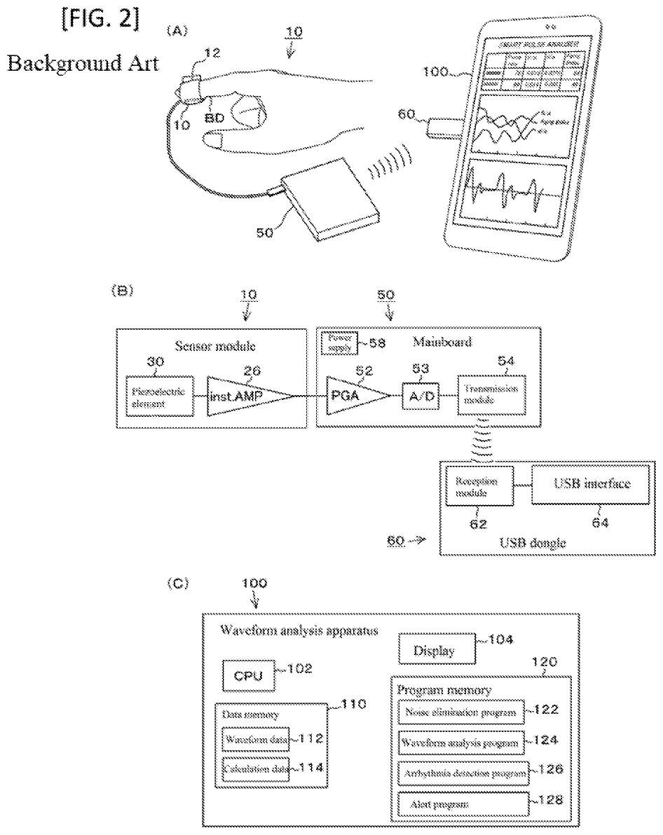

The detection signals from the piezoelectric element 30 are input to the waveform analysis apparatus 100 and analyzed, as shown in FIG. 2. To be specific, biological signals like those shown in the graph in FIG. 5(A) are detected, and a seating of the subject 400 on the toilet seat 302, as well as the subject's biological information (pulse wave information), are detected by the waveform analysis apparatus 100 shown in FIG. 2. For example, it is determined that a seating has occurred if the signal level is equal to or above a pre-set detection level. It should be noted that the detection of biological information conforms to the prior art described above. FIG. 5(B) shows that the position of the seating sensor 280 on the toilet seat 302 has been shifted by 10 cm or so toward the front edge and that the subject 400 is seated on the toilet seat 302 at an angle, so that the seating sensor 280 is no longer positioned below the thigh of the subject 400. In this case, too, the detection signals from the seating sensor 280, as shown in the graph in the same figure, allow for detection of seating and biological information regarding the subject 400. This is likely due to the vibration transmission plate 210 as it enables the pulse waves from the thigh to be transmitted to the sensor module 200 in a favorable manner.

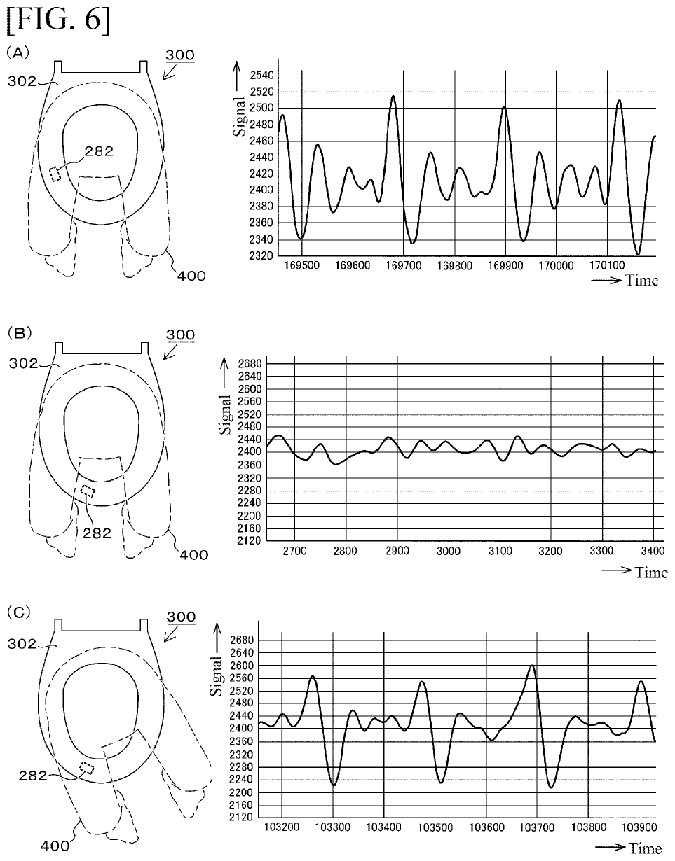

Next, an example of measurement where the vibration transmission plate 210 is not provided is explained by referring to FIG. 6. A seating sensor 282 in this example is the same as the one in FIG. 3 without the vibration transmission plate 210. (A) in the same figure, just like FIG. 5(A) described above, provides an example of measurement showing a condition where a thigh of the subject 400 is positioned directly above the seating sensor 282. As can be seen from the graph in the same figure, biological vibrations are detected in a favorable manner without the vibration transmission plate 210 when the thigh of the subject 400 is directly on the seating sensor 282.

FIG. 6(B) provides an example where the seating sensor 282 is placed on the front edge side of the toilet seat 302, where the seating sensor 282 is positioned away from the thigh of the subject 400. As can be seen from the graph in the same figure, the detected signals are very weak, making it difficult to detect seating or biological information of the subject 400. When this is compared with FIG. 5(B) described above, it is clear that providing the vibration transmission plate 210 widens the signal detection range.

On the other hand, as shown in (C) in the same figure, favorable signals are consequently obtained even when the seating sensor 280 is placed at a position similar to that in aforementioned (B), as cam be seem from the graph in the same figure, when the subject 400 is seated on the toilet seat 302 diagonally in a manner that the thigh is positioned directly on the seating sensor 280.

The foregoing shows that, according to this example, where the vibration transmission plate 210 is provided on the toilet seat 302, and where the vibration ring 40 of the sensor module 200 is contacting this vibration transmission plate 210 to allow for transmission to the piezoelectric element 30 of the pulse waves from the seated user, while the vibration transmission plate 210 is further arranged to make surface contact with the toilet seat 302, the effects of the physical size and seating position of the subject 400 can be reduced to enable detection of seating and biological information of the subject 400 in a favorable manner.

It should be noted that the present invention is not limited to the aforementioned example and that various modifications may be added to the extent that doing so does not deviate from the key points of the present invention. For example, the following are also included: (1) While the aforementioned example represents an application of the present invention to a toilet seat, it can be applied to various types of seats other than toilet seats. For example, FIG. 4(C) provides an example of applying the present invention to an automobile seat 500, where the layout is that an acrylic plate (not illustrated) is provided underneath a cushion 502 and the vibration transmission plate 210 of the seating sensor 280 makes contact with the acrylic plate. (2) While the sensor module 200 illustrated in the aforementioned example has a silicon resin 46 filled in the vibration ring 40, the silicon resin 46 may be omitted so long as doing so does not produce undesirable effects in terms of insulation, water resistance, etc. (3) The materials and shapes/dimensions of the respective parts illustrated in the aforementioned example are only an example and may be changed as deemed appropriate, if necessary. For example, while the vibration ring 40 has a circular cylinder shape in the aforementioned example, it may have a square cylinder shape or other shape as deemed appropriate. The same goes with the vibration transmission plate 210 and the fixing plate 220. (4) Human pulse wave vibrations are an example of vibrations obtained with the seating sensor 280, and it may be applied to biological vibrations of various types of seat users such as animals. (5) While both a, seating of the subject 400 and b, pulse and other biological information of the subject 400 are detected by the seating sensor 280 in the aforementioned example, solely one of the two may be detected.

### INDUSTRIAL FIELD OF APPLICATION

According to the present invention, a vibration transmission plate is provided on the seat and fixed in such a way that the vibration introducer of the sensor module contacts this vibration transmission plate; this permits widening of the seating range in which vibrations can be sensed, so that the effects of physical size and seating position can be reduced to allow for detection of seating/not-seating and biological information in a favorable manner, which is ideal for seating sensors on various types of seats such as toilet seats and automobile seats.

### DESCRIPTION OF THE SYMBOLS

10: Sensor module 12: Medical fixing tape 20: Substrate 22, 23: Electrode land 22A, 23A: Through hole 24: Ground conductor 24A, 24B: Through hole 26: Instrumentation amplifier 30: Piezoelectric element 40: Vibration ring 40A: Tip face 41: Cavity 46: Silicon resin 50: Mainboard 52: Programmable amplifier 53: Converter 54: Transmission module 60: USB dongle 62: Reception module 64: USB interface 100: Waveform analysis apparatus 104: Display 110: Data memory 112: Waveform data 114: Calculation data 122: Noise elimination program 124: Waveform analysis program 126: Arrhythmia detection program 128: Alert program 200: Sensor module 210: Vibration transmission plate 220, 230: Fixing plate 222, 232: Screw 280, 282: Seating sensor 300: Toilet bowl 302: Toilet seat 400: Subject 500: Seat 502: Cushion

1. A seating sensor structure that detects biological vibrations from a user seated on a seat, comprising:
a sensor module comprising:
a substrate;
a piezoelectric element that measures vibrations of the substrate to obtain vibration waveforms of the vibrations; and
a vibration introducer provided on the substrate, which contacts a target object and transmits vibrations of the target object to the substrate; and
a vibration transmission plate provided, as the target object, on a surface of the seat, wherein the sensor module is fixed to the seat via the vibration transmission plate in a manner that the vibration introducer contacts the vibration transmission plate, wherein the vibration transmission plate has a modulus of elasticity which is higher than a modulus of elasticity of the surface of the seat and a modulus of elasticity of the vibration introducer.

2. The seating sensor according to claim 1, wherein the vibration introducer is conductive.

3. The seating sensor according to claim 1, wherein the vibration introducer is a conductive ring and a space inside the ring is filled with a resin.

4. The seating sensor according to claim 1, wherein the vibration transmission plate makes surface contact with the seat and transmits vibrations from the user seated on the seat to the vibration introducer.

5. The seating sensor according to claim 4, wherein the modulus of elasticity of the vibration transmission plate is 70 to 2500 MPA

6. The seating sensor according to claim 1, wherein the sensor module is fixed to the seat by a fixing means in a manner that the vibration introducer is pressed against the vibration transmission plate by the fixing means.

7. A seat equipped with the seating sensor according to claim 1.

8. The seat according to claim 7, wherein said seat is a seat for a human which encompasses chairs, seats, and toilet seats.

9. A waveform analysis apparatus coupled with the sensor module of the seating sensor structure of claim 1 in a manner detecting, from detection signals of the piezoelectric element in the seating sensor, at least either one of: (i) whether or not a user of the seat is seated on the seat, and (ii) biological information of the user.
